(19) United States
(12) Patent Application Publication
(10) Pub. No.: US 2024/0255527 A1
(43) Pub. Date: Aug. 1, 2024

(54) QUANTITATIVE PARTICLE AGGLUTINATION ASSAY USING PORTABLE HOLOGRAPHIC IMAGING AND DEEP LEARNING

(71) Applicant: THE REGENTS OF THE UNIVERSITY OF CALIFORNIA, Oakland, CA (US)

(72) Inventors: Aydogan Ozcan, Los Angeles, CA (US); Hyou-Arm Joung, Los Angeles, CA (US); Yi Luo, Los Angeles, CA (US)

(73) Assignee: THE REGENTS OF THE UNIVERSITY OF CALIFORNIA, Oakland, CA (US)

(21) Appl. No.: 18/563,745

(22) PCT Filed: May 24, 2022
(86) PCT No.: PCT/US2022/030794

§ 371 Date: Nov. 22, 2023

Related U.S. Application Data
(60) Provisional application No. 63/195,648, filed on Jun. 1, 2021.

(51) Int. Cl. G01N 33/68; G01N 15/075; G01N 33/543; G06T 7/00; G06V 10/762; G06V 10/82; G06V 20/69

(52) U.S. Cl. CPC G01N 33/6893; G01N 15/075; G01N 33/54388; G06T 7/0012; G06V 10/762; G06V 10/82; G06V 20/698; G01N 2333/4737; G01N 2800/7095; G06T 2207/10056; G06T 2207/20084; G06T 2207/30004; G06T 2207/30204

(57) ABSTRACT

A quantitative particle agglutination assay device is disclosed that combines portable lens-free microscopy and deep learning for rapidly measuring the concentration of a target analyte. As one example of a target analyte, the assay device was used to test for high-sensitivity C-reactive protein (hs-CRP) using human serum samples. A dual-channel capillary lateral flow device is designed to host the agglutination reaction using a small volume of serum. A portable lens-free microscope records time-lapsed inline holograms of the lateral flow device, monitoring the agglutination process over several minutes. These captured holograms are processed, and at each frame the number and area of the particle clusters are automatically extracted and fed into shallow neural networks to predict the CRP concentration. The system can be used to successfully differentiate very high CRP concentrations (e.g., >10-500 μg/mL) from the hs-CRP range.

### RELATED APPLICATION

This application claims priority to U.S. Provisional Patent Application No. 63/195,648 filed on Jun. 1, 2021, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number 1648451, awarded by the National Science Foundation. The government has certain rights in the invention.

### TECHNICAL FIELD

The technical field generally relates to particle agglutination assays. More particularly, the technical field relates to particle agglutination assays used in point-of-care testing for antigens.

### BACKGROUND

Particle agglutination assays are widely used immunological tests based on antigen-antibody interactions. In such assays, latex particles are sensitized through the adsorption of antibodies onto their surfaces. Once the sample is introduced, the corresponding antigens attach to the antibody binding sites and the micro particles form clusters due to the target antigen's capability of binding to different sites simultaneously. The amount of agglutination between the particles is indicative of the amount of antigen present in a sample. Particle agglutination assays have been used to test for antigens in a number of bodily fluids, including e.g., saliva, urine, cerebrospinal fluid, and blood. A range of illnesses can be diagnosed using particle agglutination assays, including bacterial, fungal, parasitic and viral diseases. Its major advantages in point-of-care diagnosis include short reaction time, low sample volume, low-cost, and high specificity. The operation of conventional particle agglutination assays includes two steps. First, an operator will mix the sample of interest with a pre-processed liquid that contains antibody-coated particles. The turbidity of the mixture then changes as agglutination occurs during the mixing process. Most often, a skilled person will compare the mixture to a pre-existing standard of turbidity to give an estimation of the antigen's concentration using the naked eye. One of the barriers to its wider application lies in the assay's low sensitivity and lack of quantitative measurements. A simple practice to get semi-quantitative analysis is to perform multiple tests simultaneously with pre-diluted samples of different concentration gradients. Other research has also focused on measuring optical turbidity or light scattering with the help of a spectrometer to give quantitative readouts, which relatively complicates the system and consumes a large sample volume (e.g., 1 mL per test). Another possible solution, rather than solely measuring the turbidity, is to use optical microscopes to monitor the agglutinated particle clusters in the assay. However, such an improvement will further complicate the diagnostic system and relatively increase the cost per test.

### SUMMARY

In one embodiment, a particle agglutination assay device includes a light source configured to generate partially coherent or coherent light which illuminates a sample containing at least one antigen or target that is run through a capillary lateral flow device. Clusters of antibody-conjugated particles that are mixed with the sample are then flowed through the capillary lateral flow device. Clusters of particles cast holograms, diffraction patterns, or shadows which are captured by an image sensor over the time course of the particle agglutination assay. The device includes a microcontroller or processor configured to control operational parameters of the light source and/or image sensor. This includes, for example, the optional offloading or transfer of image files to a separate computing device for image processing. The device includes a capillary lateral flow device configured to be removably located in a sample receiving region disposed adjacent to the image sensor and interposed between the light source and the image sensor, the capillary lateral flow device includes at least one test channel coupled to at least one inlet and at least one outlet and at least one control channel coupled to at least one inlet and at least one outlet, wherein the respective outlet(s) each include at least one absorbing membrane disposed therein. Image processing software that executed by the microcontroller, another on-board processor, or a separate computer is used to back propagate images to different axial planes within the test channel(s) and control channel(s) obtained with the image sensor at a given time point in the particle agglutination assay. A series of binary black and white (B&W) masks are formed and merged (with thresholding) which is then subject to image analysis to detect the number of and total area of particle clusters at any given time point during the course of the particle agglutination assay. This information is then fed to one or more trained neural networks. In one particular embodiment, this includes two sequential trained neural networks. The first neural network is a classification network and classifies the concentration and/or type of the at least one antigen or target in the sample with a qualitative measure (e.g., high, low-measurable) or a classification decision regarding the concentration and/or type of the at least one antigen or target. Next, for the low (measurable) samples, a separate quantification neural network uses the same number and total area information from the plurality of image frames obtained over time to output a concentration of the antigen or analyte in the sample.

In another embodiment, a method of performing a particle agglutination assay for at least one antigen or target within a sample using particle agglutination assay device includes mixing the sample into a test particle solution and a control particle solution containing particles conjugated to antibodies. The test particle solution mixture is then loaded into a first inlet of a capillary lateral flow device along with the control particle solution mixture into a second inlet of the capillary lateral flow device. The capillary lateral flow device is loaded in the particle agglutination assay device. A plurality of image frames of the test channel and control channel are obtained over a period of time with an image sensor. The plurality of image frames are subject to image processing with image processing software to extract the number of particle clusters and/or area of the particle clusters in the test channel and control channel from the plurality of image frames. The number of particle clusters and area of particle clusters are then input into one or more trained neural networks configured to receive the number and/or area of particle clusters from the plurality of frames and outputs a concentration of at least one antigen or target contained in a sample and/or a qualitative output based on the concentration of the antigen.

In one embodiment, a particle agglutination assay device for measuring the concentration of at least one antigen or target within a sample includes a light source configured to generate partially coherent or coherent light along an optical path and an image sensor disposed along the optical path. The device further includes a microcontroller or processor configured to control operational parameters of the light source and/or image sensor. A capillary lateral flow device is configured to be removably located in a sample receiving region disposed along the optical path and adjacent to the image sensor, the capillary lateral flow device including at least one test channel coupled to at least one test inlet and at least one test outlet and at least one control channel coupled to at least one control inlet and at least one control outlet, wherein the at least one test outlet and the at least one control outlet include at least one absorbing membrane disposed therein, wherein the at least one test inlet is configured to receive a test particle solution and the at least one control inlet is configured to receive a control particle test solution. The image sensor acquires a time sequence of holograms or diffraction patterns generated by agglutinated particles contained within the at least one test channel and the at least one control channel over a period of time.

In another embodiment, a method of performing a particle agglutination assay for at least one antigen or target within a sample using particle agglutination assay device. The method includes providing a particle agglutination assay device that includes a light source configured to generate partially coherent light or coherent light along an optical path and an image sensor disposed along the optical path. A microcontroller or processor in the device is configured to control operational parameters of the light source and/or image sensor. A capillary lateral flow device is configured to be removably located in a sample receiving region disposed along the optical path and adjacent to the image sensor, the capillary lateral flow device including at least one test channel coupled to at least one test inlet and at least one test outlet and at least one control channel coupled to at least one control inlet and at least one control outlet, wherein the at least one test channel outlet and the at least one control channel outlet includes at least one absorbing membrane disposed therein. Image processing software is executed by the microcontroller or processor or other computing device.

To perform the assay, the sample is mixed into a test particle solution and a control particle solution containing particles conjugated to antibodies. The test particle solution mixture is loaded into the at least one test inlet and loading the control particle solution mixture into the at least one control inlet. A plurality of image frames of the at least one test channel and the at least one control channel are obtained over a period of time during the course of the particle agglutination assay with the image sensor. The plurality of image frames are subject to image processing with the image processing software to extract the number of particle clusters and/or area of particle clusters in the as a function of time during the course of the particle agglutination assay from the plurality of image frames of the at least one test channel and the at least one control channel. The number of particle clusters and/or area of particle clusters obtained as a function of time during the course of the particle agglutination assay are input into a trained neural network configured to receive the number and/or area of particle clusters from the plurality of frames and output a concentration of the at least one antigen or target contained in a sample.

### BRIEF DESCRIPTION OF THE DRAWINGS

FIG. 1A schematically illustrates a particle agglutination assay device according to one embodiment.

FIG. 1B illustrates a system that includes the particle agglutination assay device and separate computing device according to one embodiment.

FIG. 2A illustrates a photographic image of the dual-channel capillary lateral flow device in a disassembled and assembled state.

FIG. 2B illustrates a photographic image of the assembled dual-channel capillary lateral flow device.

FIG. 3A illustrates a photograph of the mobile lens-free microscopy device. (FIG. 1A) along with a schematic illustrating showing how the dual-channel capillary lateral flow device is illuminated by the light source.

FIG. 3B illustrates a photograph of the dual-channel capillary lateral flow device along with a schematic of particle agglutination inside both the test and control channels under different CRP concentration levels.

FIG. 3C illustrates the workflow or operation procedure of the quantitative particle agglutination assay.

FIG. 4A includes a photograph of the capillary lateral flow device along with a schematic drawing and lens-free microscopic images of particle clusters inside both the control and test channels at different time points.

FIG. 4B illustrates inline hologram images of the control and test channels at different time points. The processed minimum amplitude projection and particle localization results are also shown for each channel.

FIG. 5 illustrates the image processing pipeline. For each holographic image frame, the test and control channels are automatically cropped out. A multi-height digital back-propagation algorithm is applied to both channels. The extracted features are input into two shallow neural networks (Classification NN and Quantification NN) to determine the CRP concentration of the serum sample.

FIG. 6A illustrates particle area in the test channels measured at the end of the assay time (3 min) for different CRP concentration levels.

FIG. 6B is a graph of the neural network prediction of the CRP concentration inside the test channel. For the quantification neural network, the mean square error in the training, validation and testing phases were 0.252, 0.989 and 0.332 μg/mL, respectively. For the classification neural network, the training, validation and testing accuracies were 100%, 93.88% and 100%, respectively.

FIGS. 7A-7B illustrate the neural network structure and training log for the classification neural network (FIG. 7A) and the quantification neural network (FIG. 7B). The dashed vertical lines in both plots indicate the selected network model based on the minimum validation loss.

FIGS. 8A-8B: Comparison of the control channel agglutination activity according to the control particle preparation method (antigen saturated, heat-treated, and UV-exposed). Microscope images of the test and control channels for low CRP concentration samples with high false-positive agglutination (FIG. 8A), and high-CRP concentration (5.4 μg/mL) without false-positive agglutination (FIG. 8B). In the case of antigen-saturated test beads, false-positive reactions could be distinguished regardless of the CRP concentration. Heat-treated and UV-exposed particles were not distinguished from false-positive agglutination. In the case of BSA-coated particles, agglutination reaction was not observed in samples in which non-specific agglutination reaction was observed (data not shown). The heat-treated particles were prepared by incubation in a boiled water chamber for 30 min, and the UV-exposed particles were prepared by exposure under the UV lamp (6.5 mW/cm²) for 1 hour.

FIG. 9A is a graph showing the comparison of the flow rate at the capillary channels as a function of the external humidity (n=3). The flow rates were obtained within a humidity-controlled digital balance by measuring the weight change over time.

FIG. 9B is a graph showing the comparison of CRP concentration measurement results as a function of the flow rate (n=3 at 25% external humidity). CRP concentrations were measured for various sizes of absorption membrane through the quantification neural network inference, and % Bias (y-axis) represents the difference from the CRP concentration value that is measured using the optimized membrane size (circled mark: 16 mm² of absorption membrane).

FIG. 10 illustrates the time evolution of the total particle area inside the test channels for low (1.1 μg/mL) and very high (200 μg/mL) CRP concentration samples.

### DETAILED DESCRIPTION OF ILLUSTRATED EMBODIMENTS

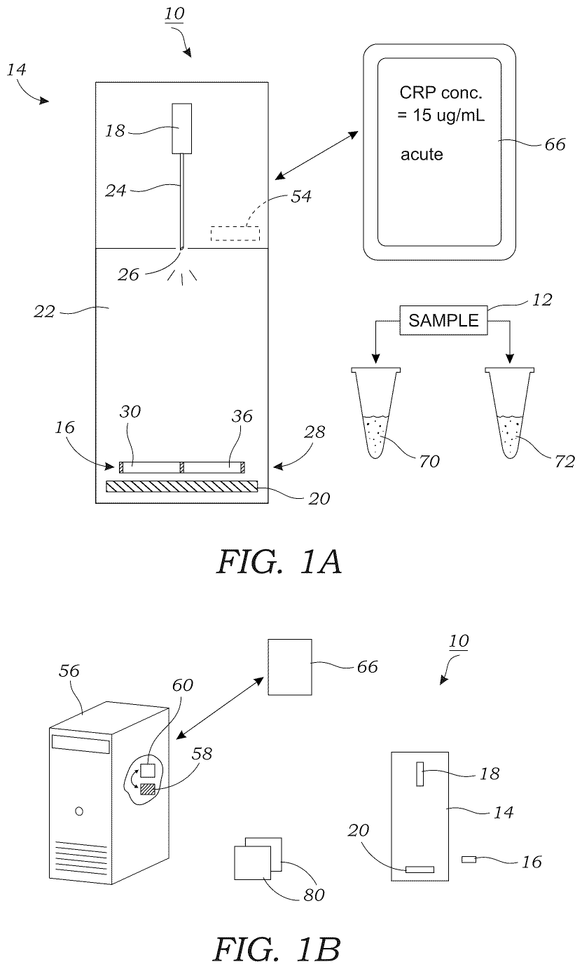

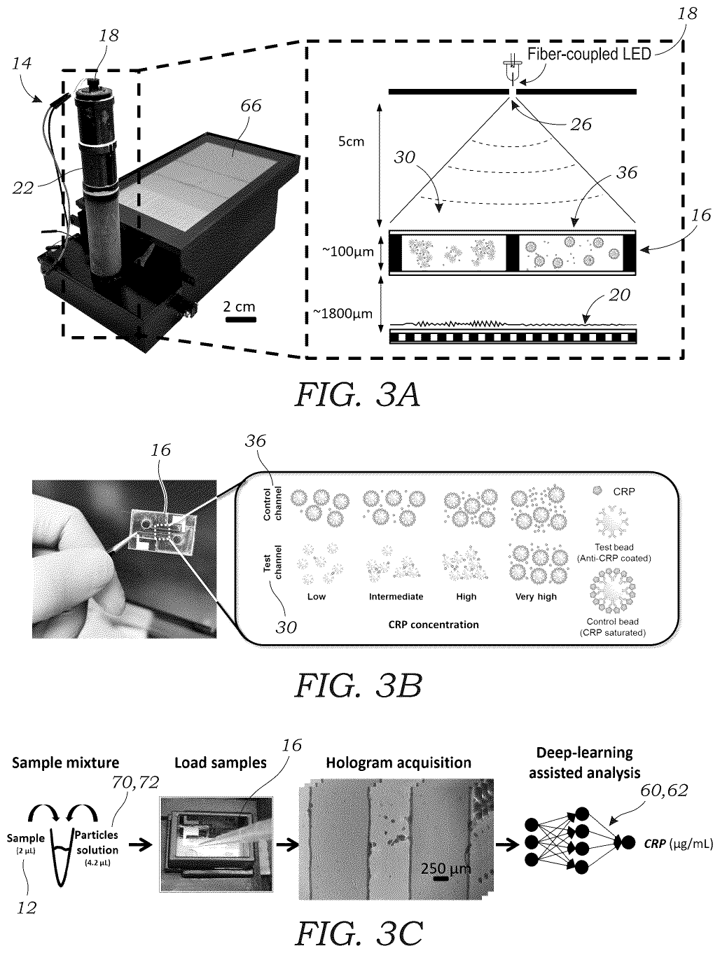

FIGS. 1A and 1B illustrate a particle agglutination assay device 10 for measuring the concentration of at least one antigen or target within a sample 12 (FIG. 1A). The particle agglutination assay device 10 includes a portable or mobile lens-free microscope 14 as best seen in FIGS. 1A, 1B, and 3A, that is used in conjunction with a capillary lateral flow device 16 that is removably inserted into the portable or mobile lens-free microscope 14 and imaged as part of the particle agglutination assay. The portable or mobile lens-free microscope 14 includes a light source 18 that is configured to generate partially coherent or coherent light along an optical path. An image sensor 20 (e.g., CMOS image sensor) is located along the optical path and is used to capture images 80 of the capillary lateral flow device 16 during the particle agglutination assay. The portable or mobile lens-free microscope 14 includes a housing or enclosure 22 that holds the light source 18 and image sensor 20. The housing or enclosure 22 also holds the capillary lateral flow device 16 during particle agglutination assay. The light source 18 may include at least one light emitting diode (LED) or at least one laser diode that illuminates at least part of the capillary lateral flow device 16 with partially coherent or coherent light. Multiple LEDs or laser diodes may also be used. In one embodiment, the LED(s) or laser diode(s) is/are coupled to an optical fiber 24 (e.g., several centimeters in length) that terminates in an end. This output end of the optical fiber 24 outputs light through an aperture 26 (e.g., 1 mm diameter) to aid in generating the partially coherent or coherent light. The image sensor 20 is located along the optical path and on an opposing side of the capillary lateral flow device 16 from the light source 18. The capillary lateral flow device 16 is thus interposed along an optical path between the light source 18 and the image sensor 20. The capillary lateral flow device 16 is located adjacent to the image sensor 20 (e.g., sample-to-sensor distance of ˜2.5 mm). The capillary lateral flow device 16 is located much further away from the aperture 26 (e.g., several centimeters) than from the image sensor 20. This enables the image sensor 20 to capture in-line holograms or diffraction patterns of particles in the capillary lateral flow device 16. The image sensor 20 is configured to take a time series of images 80 of the capillary lateral flow device 16 (e.g., over the course of the particle agglutination assay) after the sample 12 is mixed with a test particle solution 70 and control particle solution 72 and loaded into respective inlets 32, 38 in the capillary lateral flow device 16 as explained herein. For example, the image sensor 20 may capture images 80 at a frame rate of around 1 frame/per second (fps), although different frame rates are contemplated. In one embodiment, several minutes of images 80 (i.e., image frames) are obtained of the test channel 30 and control channel 36 of the capillary lateral flow device 16.

The portable or mobile lens-free microscope 14 includes a sample receiving region 28 (e.g., sample holder or sample support) that receives the capillary lateral flow device 16 for imaging with the portable or mobile lens-free microscope 14. The sample receiving region 28 may include a sample holder or sample support such as tray or substrate that holds the capillary lateral flow device 16 in the optical path while imaging takes place over time during the particle agglutination assay. The sample holder or sample support may be moveable in some embodiments so that the capillary lateral flow device 16 can be loaded/removed outside of the housing or enclosure 22 and then inserted into the housing or enclosure 22 when imaging takes place during the assay. In some embodiments, the capillary lateral flow device 16 may rest atop the image sensor 20 in which case the image sensor 20 operates as the sample holder or sample support.

The capillary lateral flow device 16 is, in one embodiment, a dual-channel capillary lateral flow device that includes a test channel 30 that is coupled to a test inlet 32 at one end and a test outlet 34 at the opposing end. Fluid flows in the direction from the test inlet 32 to the test outlet 34. The capillary lateral flow device 16 includes a control channel 36 that is coupled to a control inlet 38 at one end and a control outlet 40 at the opposing end. Fluid flows in the direction from the control inlet 38 to the control outlet 40. An absorbing membrane or pad 42 is located in the flow paths in or near the test outlet 34 and the control outlet 40. The respective absorbing membranes or pads 42 enable fluid flow within the test channel 30 and the control channel 36. The test channel 30 is used for hold the sample 12 to be tested along the test particle solution 70 while the control channel 36 channel holds the sample 12 along with a control particle solution 72. The control particle solution 72 contains particles decorated with antibodies that have already been saturated with the antigen or target so that only non-specific agglutination between the particles and unknown proteins in the sample 12 can happen and controlled for.

While the embodiment described above is a two-channel design it should be appreciated that the capillary lateral flow device 16 may have more than one test channel 30 and more than one control channel 36. In this regard, the capillary lateral flow device 16 includes at least one test channel 30 and at least one control channel 36. The at least one test channel 30 and the at least one control channel 36 are coupled to, respectively, at least one test inlet 32 and at least one control outlet 40. For example, additional test channels 30 and/or control channels 36 may be incorporated into the capillary lateral flow device 16 for multiplex testing for different analytes or targets on a single capillary lateral flow device 16. Each test channel 30 and the corresponding control channel 36 may be dedicated to a particular particle agglutination assay for different antigen or target. Alternatively, the additional test channels 30 and/or control channels 36 may be used to test different samples 12. Each of these channels 30, 36 may be coupled to their own respective inlets 32, 38 and outlets 34, 40. Absorbing membranes or pads 42 may be located in region in or adjacent to the outlets 34, 40 to drive fluid flow (or a shared absorbing membrane or pad 42 may be used across multiple outlet 34, 40).

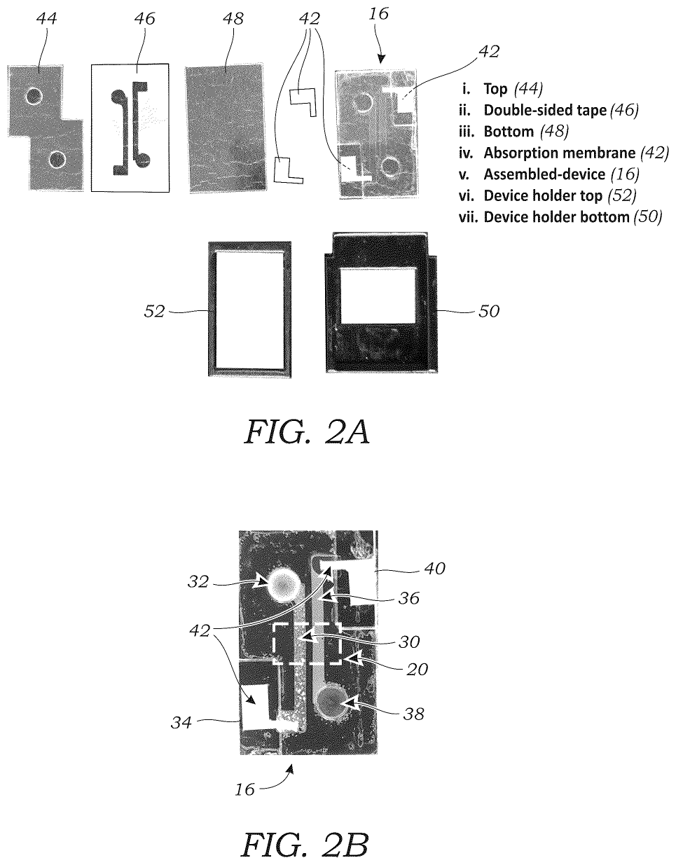

With reference to FIGS. 2A and 2B, the capillary lateral flow device 16 may be made from optically transparent materials in the region(s) where the one or more test channels 30 and the one or more control channels 36 and are located. This enables the cast holograms or diffraction patterns to be captured by the image sensor 20. The capillary lateral flow device 16 may be made from a number of layers that are sandwiched together to form a stacked structure. As one example, a first substrate layer 44 is provided that contains holes or apertures that form test inlet 32 and the control inlet 38 (e.g., top layer). A layer of double-sided tape 46 defines the test channel 30, control channel 36 along with the test inlet 32, test outlet 34, control inlet 38, and control outlet 40. The absorbing membranes or pads 42 are located in the region of the test outlet 34 and control outlet 40 and a second substrate layer 48 (e.g., bottom) is provided that is secured to the opposing side of the double-side tape 46 to form the flow path portion of the capillary lateral flow device 16 (the absorbing membranes or pads 42 are sandwiched between the two layers). The layered structure that contains the flow paths can then be optionally held by a bottom holder 50 and a top holder 52 to form the final capillary lateral flow device 16. FIG. 2B illustrates an image of the assembled capillary lateral flow device 16. Also illustrated in dashed lines is the region of the capillary lateral flow device 16 that is imaged by the image sensor 20. Both the test channel 30 and the control channel 36 are located within the field of view of the image sensor 20.

The portable or mobile lens-free microscope 14 includes a microcontroller or processing device 54 (e.g., processor) which is used to control the light source 18, image acquisition from the image sensor 20, and transfer of images 80 and/or data from the portable or mobile lens-free microscope 14. In some embodiments, images 80 captured with the portable or mobile lens-free microscope 14 are then transferred to a separate computing device 56 (e.g., FIG. 1B). The separate computing device 56 may include one or more processors 58 and image processing software 60. The image processing software 60, in one embodiment, is used to perform back propagation to one or more image planes (e.g., axial planes) located within the test/control channels 30, 36 of the capillary lateral flow device 16. The image processing software 60 may also perform particle and/or cluster detection in the image planes, feature extraction including particle/cluster count, and/or particle/cluster area. Feature extraction of the images 80 may be done at particular time points during the course of the particle agglutination assay. That is to say the timewise evolution of these extracted parameters as a function of time may be obtained. In addition, the image processing software 60 may include one or more trained neural networks 62, 64 that use the features extraction data to output concentration of an antigen contained in a sample 12 that is run through the capillary lateral flow device 16. In some embodiments, a separate trained neural network 62 is used to first classify the sample 12 based on concentration of the antigen in the sample 12 followed by a second trained neural network 64 that outputs a concentration. In this example disclosed herein this includes a high concentration of CRP (>10 μg/mL) or a lower concentration of CRP (<10 μg/mL). As an alternative to the separate computing device 56, the image processing software 60 and/or trained neural network(s) 62, 64 may be implemented by the microcontroller or other processing device 54 that is located in or associated with the portable or mobile lens-free microscope 14.

The portable or mobile lens-free microscope 14 may include a display 66 (e.g., touchscreen) that displays a Graphical User Interface (GUI) to the user. The GUI on the display 66 may be used to adjust imaging parameters, control the LED or laser diode light source 18, and data acquisition parameters (e.g., parameters of the image sensor 20 and/or microcontroller or processor 54). The display 66 may also be used to display images 80 obtained by the image sensor 20 as well as output results from the portable or mobile lens-free microscope 14. This may include qualitative and/or quantitative results of the assay. Information related to the sample 12 may also be displayed to the user on the display 66. In other embodiments, the display 66 may be incorporated on a separate device such as a mobile phone (e.g., Smartphone) that communicates with the portable or mobile lens-free microscope 14. For example, a wireless connection (e.g., Wi-Fi or Bluetooth) may connect the mobile phone to the microcontroller or processor 54. A program or application running on the mobile phone may be used to operate the portable or mobile lens-free microscope 14 as well as view results obtained thereby.

FIG. 3C illustrates the general workflow used for the particle agglutination assay. To use the portable or mobile lens-free microscope 14, a sample 12 (e.g., serum) that contains the antigen(s) or target(s) (or is suspected of containing the antigen or target) is added to a solution containing the antibody coated latex particles (test particle solution 70) to form a mixture. Examples of targets other than antigens include pathogens, bacteria, or viruses. The capillary lateral flow device 16 is first loaded with an activation buffer in both the test inlet 32 and control inlet 38. The capillary lateral flow device 16 is loaded into the portable or mobile lens-free microscope 14 and the sample 12 mixed with the test particle solution 70 containing the antibody coated latex particles and the control particle solution 72 are added into the corresponding inlets (test inlet 32 and control inlet 38). Following this, in-line hologram or diffraction pattern images 80 of the respective channels 30, 36 are then obtained over a period of time (e.g., several minutes) using the image sensor 20 under illumination from the light source 18. Different degrees of agglutination based on presence and/or different concentrations of the antigen(s) or target(s) is captured in the images 80. FIG. 3B, for example, schematically illustrates different agglutination results for low, intermediate, high, and very high CRP concentrations. These obtained images 80 are then processed as described herein. The image processing software 60 then outputs, in one embodiment, a concentration of the antigen(s) or target(s) in the sample 12. In addition, or alternatively, the image processing software 60 may classify the sample 12 based on the concentration with a qualitative output (e.g., high, low, medium, etc.). The image processing software 60 may also output a classification decision regarding the concentration of the antigen(s) or target(s) and/or the type of the antigen(s) or target(s).

### EXPERIMENTAL

A rapid and cost-effective quantitative particle agglutination assay device 10 is disclosed that uses deep learning-based analysis. As one example, FIG. 3C illustrates how the particle agglutination assay device 10 is used to automatically measure high sensitivity C-reactive protein (hs-CRP) levels in human serum samples. CRP is a general biomarker produced by the liver as a response to inflammation in the body which has a concentration in the range of up to ˜1000 μg/mL, whereas hs-CRP, in the range of 0.5 to 10 μg/mL is an indicator for the risk of myocardial dysfunction and heart failure. In the particle agglutination assay device 10, human serum samples 12 with various CRP concentrations are mixed with latex particles and the mixture is immediately loaded into a custom-designed, dual-channel capillary lateral flow device 16 (FIGS. 1A, 1B, 2A, 2B, 3A-3C, 4A). Agglutination takes place automatically while the mixture flows through the test channel 30 and control channel 36 (e.g., capillary channels) of the capillary lateral flow device 16 for ˜3 min without any further operation steps. Time-lapsed holographic images 80 of the mixture are acquired in real time with a portable or mobile lens-free microscope 14 (FIGS. 1A, 1B, 3A) to monitor and quantify the agglutination. Two neural networks 62, 64 are designed and trained to work sequentially to measure the concentration of hs-CRP and differentiate it from acute inflammation (>10-1000 μg/mL). For this, a classification network 62 is first used to identify CRP concentrations higher than 10 μg/mL and a quantification network 64 is sequentially applied specifically to hs-CRP range to predict the concentration (<10 μg/mL) of the sample. To demonstrate the success of this platform, 88 patient serum samples were measured, 65 for training and validation and 23 for blind testing, covering a CRP concentration range of 0.2 to 500 μg/mL, and achieved an R2 value of 0.912 on blind testing set with respect to the ground truth measurements, captured by an FDA-approved clinical instrument. This particle agglutination assay device 10 can be used in point of care settings to provide rapid and cost-effective measurements of various analytes.

### Materials and Methods

### Dual-Channel Capillary Lateral Flow Device Fabrication

The dual-channel capillary lateral flow device 16 is composed of different types of sheet materials (FIGS. 2A-2B). Optically clear transparent sheets (AZ42, Aztek Inc., Irvine, CA, USA) are cut into different shapes to serve as the bottom 48 and top 44 of the device 16 using a laser cutter (60W Speedy 100 CO2 laser, Trotec, USA). A double-sided tape 46 (12×12-6-467MP, 3M Inc, USA) is cut to form the side walls of both the test channel 30 and control channel 36, as well as the loading posts. The absorbing membranes or pads 42 are cut from MMM 0.8 polysulfone sheet (T9EXPPA0800S00M, Pall Corporation, USA). Before the assembly, the transparent substrate layers 44, 48 (e.g., sheets) are cleaned using an ultrasonic bath. Then, the transparent bottom piece 48, double sided tape 46, absorbing membranes or pads 42, and transparent top piece 44 are stacked together to form a sandwich-like structure.

### Preparation of Test and Control Particles

Both the test and control particle solutions 70, 72 are prepared using the CRP Latex Reagent component of the CRP Latex Test Kit (310-100, Cortezdiagnostics Inc, USA), which has an average particle diameter of 0.81 μm. To prepare the test particles, 100 μL of Latex Reagent is centrifuged at 3000 rpm for 10 min. After removal of the resulting supernatant, the beads are re-suspended in an equal volume of glycine buffer. The CRP saturated control particles are prepared by saturating antibody binding sites on the test particles; for this, the particles are diluted three times by PBS buffer and CRP antigen (30-AC10, Fitzgerald) is added to the solution to reach a final CRP concentration of 0.5 mg/mL. Following a 2-hour incubation with an orbital shaker and the addition of 1% BSA, the prepared particles are stored at 4° C.

### Assay Procedures

To perform the assay, 5 μL of the activation buffer (0.5% tween 20 in DI water) was added into both test and control channel inlets 32, 38. The channels 30, 36 were dried off and the capillary lateral flow device 16 is placed onto the CMOS image sensor 20 with a custom-designed holder. Next 2 μL of the serum sample 12 is mixed with 4.2 μL of test and control particle solutions 70, 72 individually, and they are loaded into the corresponding inlets (i.e., test inlet 32 and control inlet 38). Following this, the measurements start, recording the in-line holograms/diffraction patterns of the channels 30, 36 for 3 min.

### Collection of Clinical Samples

The use of human serum samples 12 was approved by UCLA IRB (#19-000172) for CRP testing. The CRP levels of these patient samples 12 were measured by CardioPhase hsCRP Flex® reagent cartridge (Cat. No. K7046, Siemens) and Dimension Vista System (Siemens) at UCLA Health System, which constituted the ground truth measurements.

### Mobile Lens-Free Microscope

A mobile lens-free microscope 14 was developed for monitoring of the particle agglutination assay reactions inside the capillary lateral flow device 16. A fiber-coupled light emitting diode 18 (LED, peak wavelength: 850 nm) is used to illuminate the capillary lateral flow device 16 to form inline-holograms. A CMOS image sensor 20 (IMX 219, Sony Inc.) is placed right beneath the sample holder (with a sample-to-sensor distance of˜2.5 mm) to capture the holograms at a frame rate of 1 fps. The illumination LED light source 18 and the CMOS image sensor 20 are controlled by a Raspberry Pi microcontroller 54 with a customized graphic user interface (GUI) on a display 66 that is programmed using Python.

### Particle Localization Measurements Using Multi-Height Digital Back Propagation

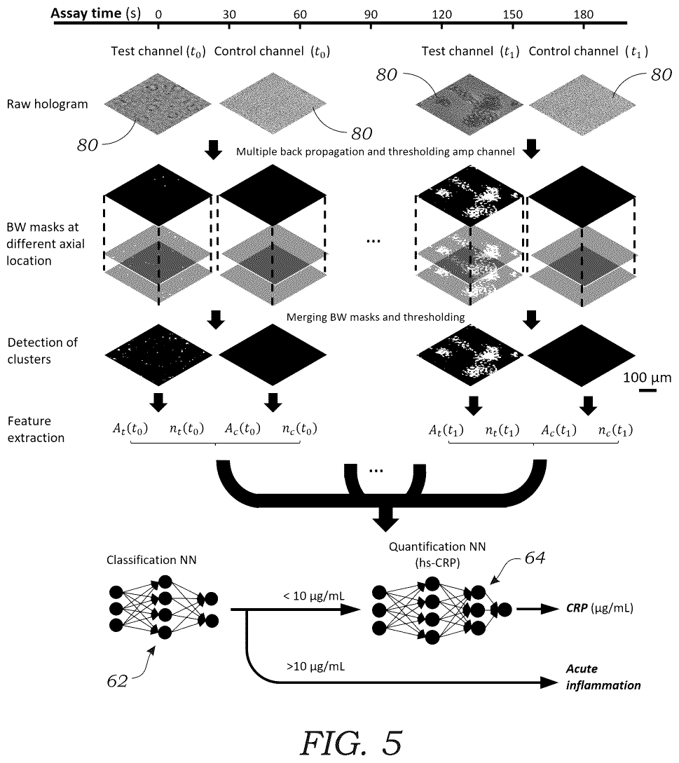

With reference to FIG. 5, the captured time-lapsed holograms are automatically processed using MATLAB image processing software 60 executed in a separate computing device 56 (although this could also be done in the portable or mobile lens-free microscope 14). The test and control channels 30, 36 are first identified and cropped out. The background of each channel 30, 36, which contains randomly located dust particles that are attached to the bottom of the capillary lateral flow device 16, is estimated using the first five frames of the image sequence. The axial distance (z₂) between the capillary lateral flow device 16 and the CMOS image sensor 20 is approximated by auto-focusing on these immobile particles. Knowing the thickness of the transparent sheet (100 μm) and the height of the channels (100 μm), the raw hologram of each channel 30, 36 is back propagated to multiple axial locations using the angular spectrum method, ranging from z₂+100 μm to z₂+200 μm with an axial step size of 10 μm. At each axial plane, a binary particle map is generated using the amplitude channel of the propagated hologram, by applying a threshold (i.e., mean amplitude value minus three standard deviations). The binary masks of ten different axial locations are summed up after removing false detections that only appeared in a single axial location. Immobile/stationary particles were also removed by comparing two consecutive frames of the binary masks. Morphological analysis is applied to this merged binary mask, which results in the estimation of the total particle area (Aₜ(t) and A_{c}(t)) and the total particle number (nₜ(t) and n_{c}(t)), as a function of time (t), in the test and control channels 30, 36, respectively.

### Neural Network Architecture

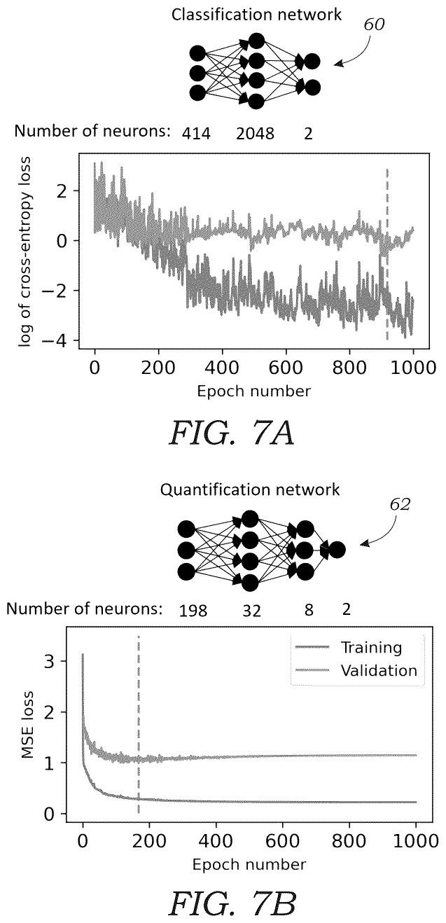

Two shallow neural networks 62, 64 (see FIGS. 5, 7A, 7B) were trained to quantitatively measure the CRP concentration over a large dynamic range. The total particle area and the total particle number in both the test and control channels 30, 36 are time-averaged with a non-overlapping sliding time-window and used as inputs to train the neural networks. The classification network 62 (FIGS. 5 and 7A) contains one fully-connected hidden layer with 2048 neurons and one output layer with 2 neurons, (O₁, O₂), which are used to indicate if the CRP concentration is below or above 10 μg/mL. Its input vector is composed of time-averaged Aₜ (t), A_{c}(t), nₜ(t) and n_{c}(t) with a window size of 1, 1, 5 and 10, respectively. A cross-entropy loss function (L₁) is used to calculate the error gradients used in the training phase:

$${L}_{1} = \frac{1}{N} {\sum }_{i = 1}^{N} - ( {y}_{i} log ( {p}_{i} ) + ( 1 - {y}_{i} ) log ( 1 - {p}_{i} ) ) \qquad ( 1 )$$

where yᵢ is a binary indicator (the ground truth label), representing if the measured CRP concentration is above 10 μg/mL or not, for each measurement i, in a training batch of N different measurements. pᵢ indicates the probability whether the CRP concentration is higher than 10 μg/mL or not for a given measurement i. It is calculated using the output values of the network O=[O₁, O₂] as

$$p = \frac{exp ( {O}_{1} )}{exp ( {O}_{1} ) + exp ( {O}_{2} )} \qquad ( 2 )$$

The quantification network 64 (FIGS. 5, 7B), on the other hand, contains two fully-connected hidden layers with 32 and 8 neurons separately, and one output layer with a single neuron (Q), outputting the predicted CRP concentration within the hs-CRP range. The time-averaging window sizes for Aₜ(t), A_{c}(t), nₜ(t) and n_{c}(t) are equal to 30, 30, 1 and 30, respectively. A mean-square-error loss function (L₂) is applied for the training of the quantification network 64, defined as:

$${L}_{2} = \frac{1}{N} {\sum }_{i = 1}^{N} \sqrt{{( {Q}_{i} - {C}_{i} )}^{2}} \qquad ( 3 )$$

In Eq. (3), Qᵢ is the value of the single output neuron, representing the predicted CRP concentration, and Cᵢ is the ground truth concentration measured by the gold standard instrument for each measurement i.

The hyper-parameters of both neural networks 62, 64 (e.g., the number of neurons and sliding window sizes for Aₜ(t), A_{c}(t), nₜ(t) and n_{c}(t)) are optimized through a greedy search. For each parameter search, the candidates were selected from a predefined list. For example, in the search list for the quantification network 64, the number of neurons for the first (second) hidden layer included 32, 64, 128 and 256 (8, 16, 32 and 64) as selection options. Similarly, for the classification network 62, the number of neurons for the hidden layer (only one) included 128, 256, 1024 and 2048. For the sliding window size, the search list included 1, 5, 10, 15 and 30. For each point of the greedy search, the corresponding neural network 62, 64 was trained for 5 times with 500 epochs in each training, using the Adam optimizer with a learning rate of 10⁻⁴.

The validation loss was then averaged to find the best candidates. After optimizing all the hyper-parameters, both networks 62, 64 were trained using the Adam optimizer for 1,000 epochs. At the beginning, the learning rate was set to 10⁻⁴. The validation loss was calculated after every epoch of training and a learning rate scheduler was adopted to monitor the validation loss so that the learning rate was reduced by a factor of two if there was no improvement in 100 consecutive epochs of training. The training, validation and testing datasets of the classification network 62 had 96, 49 and 44 different measurements, respectively, and the quantification network 64 was trained with training, validation and testing datasets composed of 71, 31 and 33 different measurements, respectively. The networks 62, 64 were composed using Pytorch and trained on a desktop computer (Origin PC Corp., FL, US) using a CPU only. The typical training time for classification and quantification networks 62, 64 is ˜30 sec and ˜60 sec, respectively. For blind inference, the classification and quantification neural networks 62, 64 on average took less than 0.1 ms per test using a desktop computer with 64 GB memory and i9-7900X CPU (Intel corp., CA, US).

### Results

### Quantitative Particle Agglutination Assay and Portable Holographic Reader

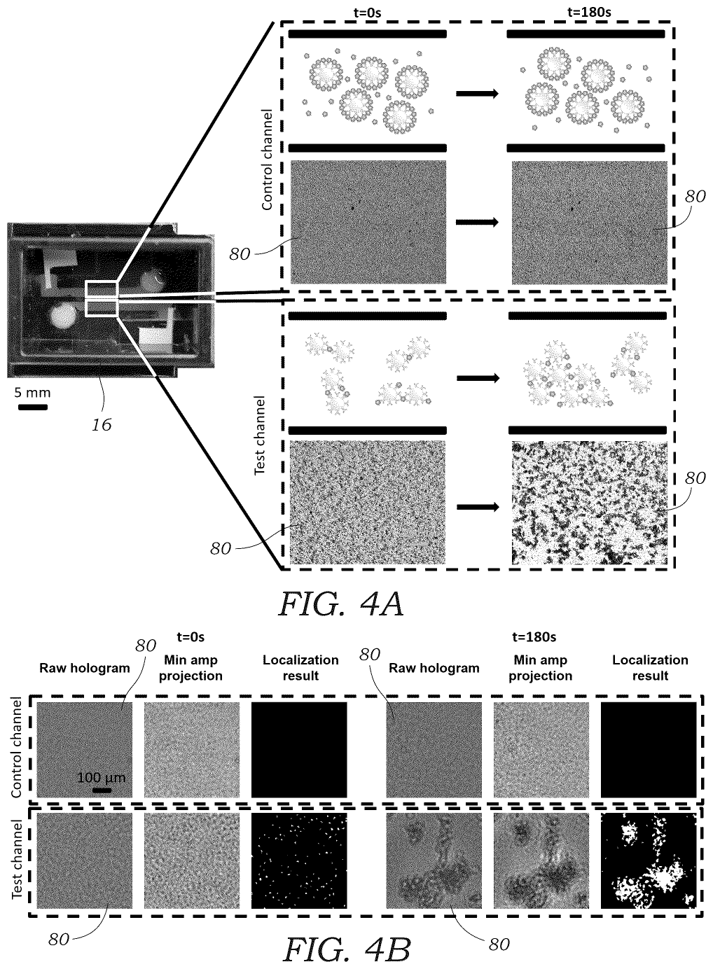

A lateral flow particle agglutination assay device 10 was developed to quantitatively measure the CRP concentration of serum samples 12 by monitoring the particle agglutination reaction between CRP and antibody coated latex particles. The assay was composed used custom-designed, low-cost dual-channel capillary lateral flow device 16 to host the antigen-antibody interactions and a portable or mobile lens-free microscope 14 to monitor and quantify the agglutination process (FIGS. 3B, 4A, 4B). The operation principles of the lateral flow-based particle agglutination assay are depicted in FIG. 3C. Two microliters of human serum sample 12 under test is mixed with test beads (antibody-coated latex micro-particles) and control beads (CRP saturated particles) separately, i.e., 4 μL of serum sample 12 is consumed per test. Without any incubation, the mixtures are directly loaded into the test and control channels 30, 36 of the dual-channel capillary lateral flow device 16. The device 16 is custom-designed and fabricated using low-cost sheet materials, with a total material cost of 1.79 ¢ per chip (Table 1), which can be further reduced through mass-production and economies of scale (total assay cost: $20.8 ¢/test including test bead and CPR antigen cost).

TABLE 1

| Materials | Vender | Cat. No. | Cost/Test (¢) |
|---|---|---|---|
| Absorption | Pall Corp. | T9EXPPA0800S00M | 0.74 |
| membrane | | | |
| Transparency | Aztek | AZ-42 | 1.04 |
| Film/Substrates | | | |
| Double-Sided Tape | Digikey | 3M9726-ND | 0.02 |
| | | Total Cost | 1.79 |

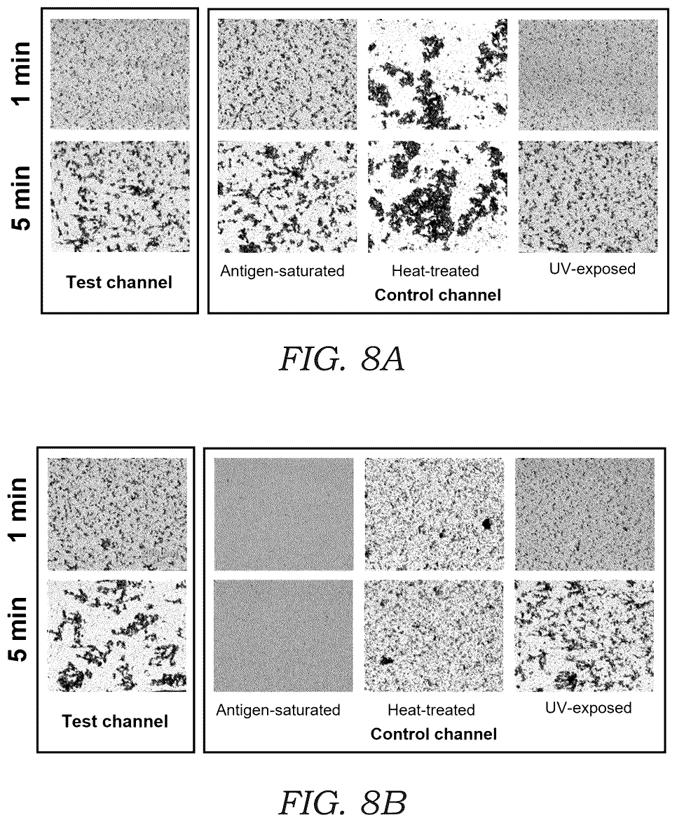

A sheet-tape-sheet sandwich structure was manually assembled to form capillary lateral flow device 16 with the test channel 30 and the control channel 36. An absorption membrane or pad 42 was inserted at the test outlet 34 and the control outlet 40. Water evaporation on the membrane or pad 42 provides the driving force for continuous flow. The diffusion of both CRP and latex particles in the laminar flow enabled the antigen-antibody reaction in the test channel 30, resulting in agglutinated particle clusters with their size varying as a function of the test time and the CRP concentration in serum (FIG. 4A). In the control channel 36, however, given that the antibodies have already been saturated with CRP (by design), only non-specific agglutination between latex particles and unknown proteins in serum can happen (FIGS. 8A-8B). The incorporation of the control channel 36 in this lateral flow device design provides a self-calibration tool for mitigating potential false positive agglutination.

During the total assay time (3 min), time-lapsed inline holograms were acquired using the portable or mobile lens-free microscope 14 at 1 frame/sec. The captured holograms (FIG. 4B) were automatically processed by the image processing software 60 and for each frame, the locations of the test and control channels 30, 36 were digitally cropped out. Given the existence of a large number of particles whose diameter is close to the illumination wavelength, the liquid mixtures in both channels 30, 36 are highly scattering. To localize and measure the forming particle clusters, a multi-height back-propagation algorithm was applied to each channel 30, 36 and the total particle number as well as the total particle area were extracted (FIG. 5). Time series of these features were utilized to determine the CRP concentration of the serum sample 12 using two sequentially collaborating, trained neural networks 62, 64 (FIG. 5), i.e., a classification network 62 and a concentration quantification network 64, respectively. Both of these neural networks 62, 64 are shallow with a low number of trainable parameters, to ensure rapid inference speed and avoid overfitting (FIGS. 7A-7B). While total particle number and total particle area were extracted and used for the trained neural networks 62, 64, in some embodiments, the results may be obtained with just the total particle number or the total particle area.

### Quantification of CRP Concentration Using Deep Learning

88 human serum samples 12 were collected from different patients with various CRP concentrations. 144 different measurements were conducted on these clinical samples 12 (duplicate measurements were conducted on 56 samples). Given that only three out of 88 serum samples 12 had CRP concentrations higher than 10 μg/mL, additional acute inflation samples were created by spiking CRP into clinical samples 12 to achieve a concentration of >10 μg/mL. For this purpose, three clinical samples 12 with original CRP concentrations lower than 0.2 μg/mL were spiked to achieve five different CRP concentrations (20, 50, 100, 200 and 500 μg/mL), forming 15 additional samples 12 to represent a concentration range of >10 μg/mL. A total of 45 measurements were performed on these CRP-spiked, additional samples (triplicate measurements on each sample). Therefore, the total number of CRP measurements that have been made with the assay platform is 144+45=189.

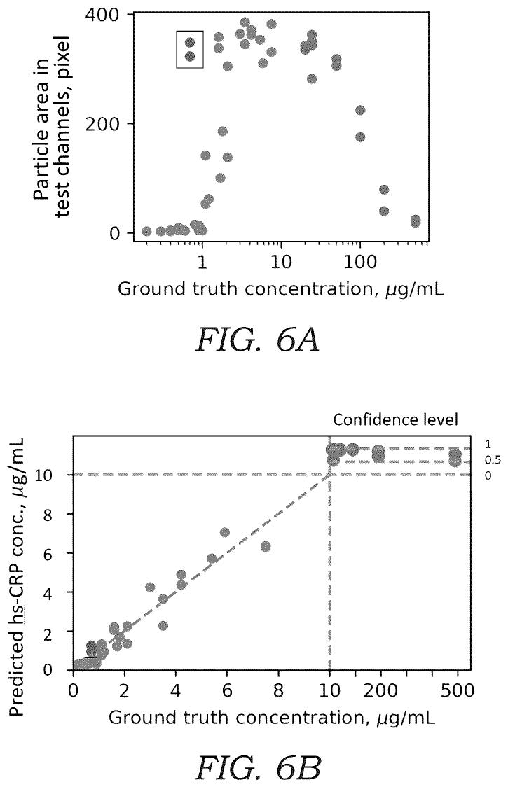

Conventional particle agglutination assays suffer from false-negative diagnosis of high concentration cases due to the saturation of antibody's binding sites by excessive antigens, also as known as the hook effect. The impact of the hook effect can also be clearly seen in the raw measurements. The total cluster size measured at the end of the assay time in the test channel 30 of the serum samples with different CRP concentrations is illustrated in FIG. 6A. The cluster size increased with increasing concentration of CRP, until it reached a maximum at around 10 μg/mL. Further increases of CRP concentration saturated the antibody's binding sites and reduced the cluster size (FIG. 6A). To overcome the hook effect and accurately identify the unknown CRP concentration over a large dynamic range of CRP concentrations, two different neural networks 62, 64 were designed to work sequentially (FIG. 5). The first one, i.e., the classification network 62, was designed to distinguish if a sample 12 has a CRP concentration higher than 10 μg/mL or not. The network 62 was trained (and validated) using 96 (49) different measurements out of all 189 measurements, covering CRP concentrations ranging from 0 to 500 μg/mL. 44 measurements from 23 different patients that were not used in the training and validation datasets were used to form the testing dataset. The second network 64, i.e., the quantification network 64, was designed to quantitatively determine the hs-CRP concentration in the sample 12 (covering 0-10 μg/mL). The network 64 was trained using the same measurement dataset separation reported earlier, but eliminating all the measurements on samples with a CRP concentration that is higher than 10 μg/mL. As a result, the training, validation and testing sets of the quantification network 64 contained 71, 33 and 31 different measurements, respectively.

The decision-making performance of this two-network based computational sensing system is depicted in FIG. 6B. For hs-CRP, the predicted concentration by the device is plotted with respect to the corresponding ground truth concentration measured for each sample 12 at UCLA Health System. The dashed line indicates a perfect prediction, i.e., y=x. For samples with high CRP concentration (>10 μg/mL), the confidence level of the classification result is also presented in the same plot. These results reveal that the classification network 62 successfully separated all the samples 12 in the blind testing dataset based on their concentration (acute inflammation vs. hs-CRP), overcoming the hook effect. With 31 blind tests quantified in the hs-CRP range, the R² value was found to be 0.912 with respect to the y=x line, demonstrating the inference success of the quantification neural network 64.

To further highlight the capabilities of this neural network-based inference of the target analyte concentration, in FIG. 6A measurement results are reported on a clinical sample 12 with 0.7 μg/mL of CRP measured by the gold standard instrument. At the end of the assay time (180 sec), the total particle area in the test channel 30 provided a strong false-positive signal (two dots in FIG. 6A surrounded by rectangle), which can occur frequently in clinical testing due to unknown proteins present in patient serum. Even for these challenging tests that would normally result in a false positive, the quantification neural network successfully inferred the CRP concentration in these duplicate measurements as indicated with the dots (surrounded by rectangle) in FIG. 6B, avoiding a false cardiovascular risk factor classification, which exemplifies the inference power of the imaging-based particle agglutination assay.

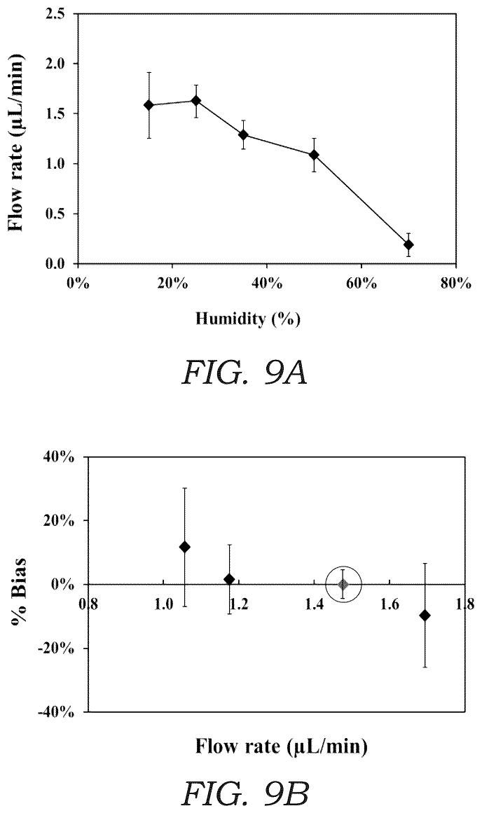

In the particle agglutination assay device 10, the antibody-antigen interaction is assisted by the laminar flow inside the dual-channel capillary lateral flow device 16. The flow rate is a key parameter to guarantee the stable reactivity of the assay. The size of the absorption membrane or pad 42 and the external humidity are critical factors in determining the flow rate inside the capillary channels 30, 36. The membrane or pad size that was used herein was optimized by evaluating the assay's reactivity under different flow rates and different humidity conditions (see FIGS. 9A-9B). Through these optimization experiments, a membrane of size of 16 mm² was selected which resulted in an average flow rate of 1.45±0.3 L/min. In addition, it was confirmed that the selected membrane or pad 42 operates stably at all the tested humidity levels (10-50%) except for 70%, through the corresponding comparisons of the reactivity and humidity evaluation results.

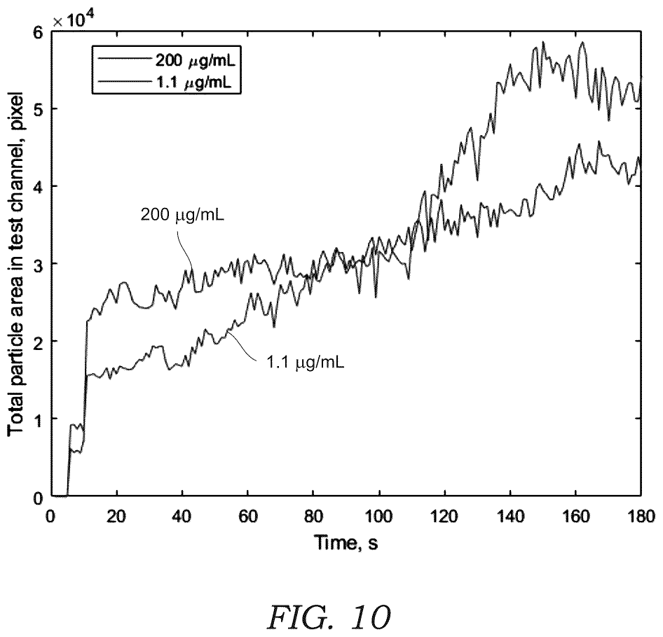

Several studies were reported in the literature to overcome the hook effect in sensor response by using advanced assay designs. Compared to conventional particle agglutination assays, this platform provides kinetic information of the agglutination process, which is essential in overcoming the hook effect. Although being similar at the end of the entire assay time, the total particle/cluster area in the test channel 30 for serum samples 12 with low and very high CRP concentrations present different dynamic patterns as a function of time, tracked with the time-lapse holographic imaging system (FIG. 10). Agglutination gradually occurred in low CRP samples 12, showing a slow increase in particle cluster area as a function of time. On the other hand, very high concentration CRP samples 12 quickly saturated the binding sites of the antibodies on the latex particles in the first few seconds of the assay, and the imaged particle area stayed stable in the remaining assay time.

It is important to emphasize the importance of the neural network models 62, 64. The raw measurements of the total particle area and total particle number in both the test and control channels 30, 36 are not easy to understand or interpret. To better illustrate the importance of the neural networks 62, 64 employed herein, the same data was used as input into an L1-norm regularization algorithm (i.e., least absolute shrinkage and selection operator, LASSO) to perform the same analyte classification and quantification tasks using human serum samples. On the blind testing dataset, the classification LASSO scored an accuracy of 82.61% and the quantification LASSO achieved an R² value of 0.3741; this poor inference performance of LASSO further emphasizes the necessity and advantages of using neural networks 62, 64, in the computational sensing platform.

In terms of digital processing of these spatio-temporal changes within the test channel 30, the particle localization algorithm that was employed significantly simplified the neural network structure. Although the raw acquired hologram images 80 were noisy, after the particles were localized using the multi-height back-propagation of each hologram, a shallow neural network 62, 64 architecture with a small number of neurons and trainable parameters was sufficient to quantify and classify the CRP concentration of serum samples over a large dynamic range. This shallow network architecture also shortened the inference time through each one of the networks 62, 64: on average it took less than 0.1 ms per CRP test to have an output from the classification and quantification neural networks 62, 64. With batch processing of multiple tests in parallel, this inference time can be further reduced.

A rapid, simple, and cost-effective particle agglutination assay device 10 is disclosed for point-of-care testing by using a custom-designed capillary lateral flow device 16 and a mobile lens-free microscope 14. The agglutination of particles was captured, as a function of time, by a mobile lens-free microscope 14 and digitally processed by two different neural networks 62, 64 for classification and quantification of the CRP concentration of the serum sample 12 under test. This deep learning-assisted sensor has a low material cost (1.79 ¢/test) and requires a small sample volume (4 μL of serum per test), presenting a promising platform for various point-of-care sensing applications.

While embodiments of the present invention have been shown and described, various modifications may be made without departing from the scope of the present invention. For example, while the particle agglutination assay device 10 was used for CRP, it should be appreciated that a wide variety of antigens or targets may be analyzed in a similar manner. This includes other disease biomarkers (e.g., using a molecule or set of molecules associated with the particular disease). The particle agglutination assay may also be used to determine the concentration of or identity of a type of pathogen, bacterium, or virus using a molecule or set of molecules that correspond to the particular type of pathogen, bacterium, or virus. The capillary lateral flow device 16 may include multiple test channels 30 and/or control channels 36 with different channels directed to different antigens, targets, pathogens, bacteria, or viruses (or different samples 12). The invention, therefore, should not be limited, except to the following claims, and their equivalents.

1. A particle agglutination assay device for measuring the concentration of at least one antigen or target within a sample comprising:
a light source configured to generate partially coherent or coherent light along an optical path;
an image sensor disposed along the optical path;
a microcontroller or processor configured to control operational parameters of the light source and/or image sensor;
a capillary lateral flow device configured to be removably located in a sample receiving region disposed along the optical path and adjacent to the image sensor, the capillary lateral flow device comprising at least one test channel coupled to at least one test inlet and at least one test outlet and at least one control channel coupled to at least one control inlet and at least one control outlet, wherein the at least one test outlet and the at least one control outlet comprise at least one absorbing membrane disposed therein, wherein the at least one test inlet is configured to receive a test particle solution and the at least one control inlet is configured to receive a control particle test solution; and
wherein the image sensor acquires a time sequence of holograms or diffraction patterns generated by agglutinated particles contained within the at least one test channel and the at least one control channel over a period of time.

2. The particle agglutination assay device of claim 1, wherein the light source comprises at least one light emitting diode or at least one laser diode.

3. The particle agglutination assay device of claim 2, further comprising an aperture and/or fiber optic cable interposed between the image sensor and the light source.

4. The particle agglutination assay device of claim 1, wherein the microcontroller or processor executes image processing software configured to back propagate raw hologram image frames obtained by the image sensor at a given time point in the particle agglutination assay to a plurality of axial planes located within the at least one test channel and the at least one control channel, wherein the back propagated images within the at least one test channel and the back propagated images within the at least one control channel are respectively merged and subject to thresholding to identify clusters of particles in the at least one test channel and the at least one control channel at the given time point in the particle agglutination assay.

5. The particle agglutination assay device of claim 4, wherein the image processing software is further configured to extract the number and/or area of the particle clusters as a function of time during the course of the particle agglutination assay.

6. The particle agglutination assay device of claim 5, further comprising a first trained neural network configured to receive the number and/or area of particle clusters from a plurality of frames captured as a function of time during the course of the particle agglutination assay and output a qualitative measure or a classification decision regarding the concentration and/or type of the at least one antigen or target in the sample.

7. The particle agglutination assay device of claim 6, further comprising a second trained neural network configured to receive the number and/or area of particle clusters from a plurality of frames captured as a function of time during the course of the particle agglutination assay and output a quantitative measure of concentration of the at least one antigen or target in the sample.

8. The particle agglutination assay device of claim 1, further comprising a computing device configured to receive hologram image frames as function of time obtained during the course of the particle agglutination assay from the microcontroller or processor, the computing device executes imaging processing software configured to back propagate each one of the raw hologram image frames obtained by the image sensor to a plurality of axial planes located within the at least one test channel and the at least one control channel, wherein the back propagated images with the at least one test channel and the back propagated images within the at least one control channel are respectively merged and subject to thresholding to identify clusters of particles forming in the at least one test channel and the at least one control channel as a function of time during the course of the particle agglutination assay.

9. The particle agglutination assay device of claim 8, wherein the image processing software is further configured to extract the number and/or area of particle clusters in each image frame as a function of time during the course of the particle agglutination assay.

10. The particle agglutination assay device of claim 9, further comprising one or more neural networks configured to receive the number and/or area of particle clusters from a plurality of frames captured as a function of time during the course of the particle agglutination assay and output a concentration of the at least one antigen or target contained in the sample.

11. A method of performing a particle agglutination assay for at least one antigen or target within a sample using particle agglutination assay device:
providing a particle agglutination assay device comprising:
a light source configured to generate partially coherent light or coherent light along an optical path;
an image sensor disposed along the optical path;
a microcontroller or processor configured to control operational parameters of the light source and/or image sensor;
a capillary lateral flow device configured to be removably located in a sample receiving region disposed along the optical path and adjacent to the image sensor, the capillary lateral flow device comprising at least one test channel coupled to at least one test inlet and at least one test outlet and at least one control channel coupled to at least one control inlet and at least one control outlet, wherein the at least one test channel outlet and the at least one control channel outlet comprise at least one absorbing membrane disposed therein; and image processing software executed by the microcontroller or processor or other computing device;
mixing the sample into a test particle solution and a control particle solution containing particles conjugated to antibodies;
loading the test particle solution mixture into the at least one test inlet and loading the control particle solution mixture into the at least one control inlet;
obtaining a plurality of image frames of the at least one test channel and the at least one control channel over a period of time during the course of the particle agglutination assay with the image sensor;
subjecting the plurality of image frames to image processing with the image processing software to extract the number of particle clusters and/or area of particle clusters in the as a function of time during the course of the particle agglutination assay from the plurality of image frames of the at least one test channel and the at least one control channel; and
inputting the number of particle clusters and/or area of particle clusters obtained as a function of time during the course of the particle agglutination assay into a trained neural network configured to receive the number and/or area of particle clusters from the plurality of frames and output a concentration of the at least one antigen or target contained in a sample.

12. The method of claim 11, wherein the trained neural network comprises two trained neural networks arranged in series, with a first trained neural network configured to output a qualitative measure of or a classification decision regarding the concentration and/or type of the at least one antigen or target in the sample and a second trained neural network configured to output a quantitative measure of concentration of the at least one antigen or target in the sample.

13. The method of claim 11, wherein the at least one antigen or target comprises C-reactive protein (CRP).

14. The method of claim 11, wherein the at least one antigen or target comprises a disease biomarker or a specific molecule or set of molecules.

15. The method of claim 11, wherein the at least one antigen or target comprises a molecule or set of molecules corresponding to a type of pathogen, a type of bacterium, or a type of virus.

16. The method of claim 11, wherein the capillary lateral flow device comprises a plurality of test channels, with each test channel configured for a different antigen or target.

17. The method of claim 12, wherein the at least one antigen or target comprises C-reactive protein (CRP).

18. The method of claim 12, wherein the at least one antigen or target comprises a disease biomarker or a specific molecule or set of molecules.

19. The method of claim 12, wherein the at least one antigen or target comprises a molecule or set of molecules corresponding to a type of pathogen, a type of bacterium, or a type of virus.

20. The method of claim 12, wherein the capillary lateral flow device comprises a plurality of test channels, with each test channel configured for a different antigen or target.
